(19) United States
(12) Patent Application Publication
(10) Pub. No.: US 2025/0302654 A1
(43) Pub. Date: Oct. 2, 2025

(54) INTERCHANGEABLE, MODULAR JOINT ASSEMBLY FOR ORTHOTIC DEVICES

(71) Applicant: HiTek Fabrication, St. Peters, MO (US)

(72) Inventor: Shawn Bright, Lake Saint Louis, MO (US)

(21) Appl. No.: 19/069,919

(22) Filed: Mar. 4, 2025

Related U.S. Application Data
(60) Provisional application No. 63/571,601, filed on Mar. 29, 2024.

(51) Int. Cl. A61F 5/01; A61F 2/60

(52) U.S. Cl. CPC A61F 5/0125; A61F 2/604; A61F 2005/0155

(57) ABSTRACT

A modular joint assembly for a brace device for a joint is provided. The modular joint assembly can include an elongated upper arm that can be fastened to an upper leg strut of the brace device, and an elongated lower arm that can be fastened to a lower leg strut of the brace device. The upper arm and the lower arm can be connected with each other at a pivot axis about which the upper arm and the lower arm pivot relative to each other. One or both of the upper arm or the lower arm can be separated from another of the upper arm or the lower arm and replaced with a longer or shorter arm to change a location of the pivot axis in the brace device.

### CROSS-REFERENCE TO RELATED APPLICATIONS

This application claims priority to U.S. Provisional Application No. 63/571,601 (filed 29 Mar. 2024), the entire disclosure of which is incorporated herein by reference.

### BACKGROUND

### Technical Field

The subject matter described herein relates to orthotic devices or braces, such as knee braces and ankle braces having vertical struts connected with each other by hinged joints.

### Discussion of Art

Orthotic devices, or orthoses, are medical devices that can include braces that mechanically support, align, and improve function of joints, such as knee and ankle joints. The orthotic braces used for knees can be referred to as knee, ankle, foot (FAFO) braces and the orthotic devices used for ankles can be referred to as ankle foot (AFO) braces.

Orthotic braces may include vertical struts that are connected by a hinged joint. The hinged joint may be located along a center of the knee or ankle so that the hinged joint can pivot about one or more axes as the patients wearing the braces walk. The vertical struts may include an upper pair of opposed struts and a lower pair of opposed struts. These struts may be bent to follow the contours of the patient's thigh above the knee and shin below the knee for a knee orthotic braces, and bent to follow the contours of the patient's foot and leg above and below the ankle for ankle orthotic braces.

Bending the struts to match the contours of a patient's body may be required to ensure proper fit and operation of the braces. Otherwise, the braces may not assist the patient and may hinder movement of the patient due to misalignment of the hinged joint due to the improperly fit struts. As patients grow, especially children wearing knee braces, struts may need to be re-shaped or replaced often to match changes in the shapes (e.g., lengths) of the children's legs as the children grow.

But changing the shape of struts may not be possible, or may be very difficult and time-consuming. For example, the struts may be formed from a metal, metal alloy, or composite material that is difficult to bend into another shape that exactly matches the contours of a patient's leg. Additionally, these types of struts are more prone to breaking if bent too frequently. Other struts may be formed from polymers that cannot be bent into different shapes after being formed without damaging or breaking the struts. Additionally, even if the struts can be re-bent to match a changing shape of a patient's leg, re-bending the struts is a time-consuming and laborious manual process. This process can take days or weeks due to backups in work to complete, during which time the patient may be unable to walk without the brace.

Additionally, there are different types of hinged joints that connect the struts. For example, one hinged joint is a drop lock or lock knee joint having two pivoting arms connected with each other to rotate about a pivot axis. A slidable ring can move up and down the length of the upper strut and the arm of the knee joint that is coupled with the upper strut. If the knee joint is straightened (e.g., when a patient extends and straightens their knee) while the patient is standing, gravity can pull the ring down the upper strut to a position that extends around both the upper arm of the knee joint (that is connected to the upper strut) and the lower arm of the knee joint (that is connected to the lower strut). In this position, the ring can lock the arms of the knee joint in position such that the knee joint can no longer pivot as the arms are constrained from pivoting relative to each other by the ring. This can be used to assist patients in keeping their leg straight (e.g., for support while standing). Other types of knee joint includes a spring assisted joint having a spring that exerts a force to assist the patient in straightening their knee from a bent position, a free motion joint that does not lock or assist the patient's movements, a knee joint having a ratchet that can temporarily lock the knee joint at different pivot angles between the arms of the knee joint, etc.

Patients may want to use different knee joints in different situations. Currently known braces, however, may require the patient to bring the brace to a healthcare provider to have the entire brace or most of the brace re-fit to the patient to replace the knee joint. Stated differently, current braces do not allow for the easy swapping out of joints. This can be frustrating to patients and limit activities of the patients.

### BRIEF DESCRIPTION

In one example, a modular joint assembly for a brace device for a joint is provided. The modular joint assembly can include an elongated upper arm that can be fastened to an upper leg strut of the brace device, and an elongated lower arm that can be fastened to a lower leg strut of the brace device. The upper arm and the lower arm can be connected with each other at a pivot axis about which the upper arm and the lower arm pivot relative to each other. One or both of the upper arm or the lower arm can be separated from another of the upper arm or the lower arm and replaced with a longer or shorter arm to change a location of the pivot axis in the brace device.

In another example, a kit for adjusting a brace device for a joint is provided. The kit can include plural modular joint assemblies each having an elongated upper arm that can be fastened to an upper leg strut of the brace device and an elongated lower arm that can be fastened to a lower leg strut of the brace device. The upper arm and the lower arm can be connected with each other at a pivot axis about which the upper arm and the lower arm pivot relative to each other. Each of the modular joint assemblies in the kit can have a differently sized upper arm, a differently sized lower arm, or both the differently sized upper arm and the differently sized lower arm than the other modular joint assemblies in the kit. One or both of the upper arm or the lower arm of each of the modular joint assemblies can be separated from another of the upper arm or the lower arm of the same modular joint assembly and replaced with a longer or shorter arm of another one of the modular joint assemblies to change a location of the pivot axis in the brace device.

In another example, a method is provided that includes disconnecting a first upper arm or a first lower arm of a first modular joint assembly in a brace device for a joint from an upper strut or a lower strut of the brace device. The upper strut can be connected to an upper leg cuff and the lower strut can be connected to a lower leg cuff. The method also can include obtaining a longer or shorter second upper arm or a second lower arm, and connecting the longer or shorter second upper arm or the second lower arm to the upper strut or the lower strut to change a location of a pivot axis of the first modular joint assembly or maintain alignment of the pivot axis of the first modular joint assembly with a rotation axis of a joint in the brace device.

### BRIEF DESCRIPTION OF THE DRAWINGS

The subject matter may be understood from reading the following description of non-limiting embodiments, with reference to the attached drawings, wherein below:

FIG. 1 illustrates a side view of one example of an orthotic knee brace device having a modular joint assembly;

FIG. 2 illustrates an opposite side view of the brace device shown in FIG. 1 with another modular joint assembly;

FIG. 3 illustrates one example of a ratcheting modular joint assembly shown in FIG. 1;

FIG. 4 illustrates one example of a drop lock modular joint assembly shown in FIG. 2;

FIG. 5 illustrates one example of a set or kit of modular joint assemblies; and

FIG. 6 illustrates a flowchart of one example of a method for fitting a brace device to a patient.

### DETAILED DESCRIPTION

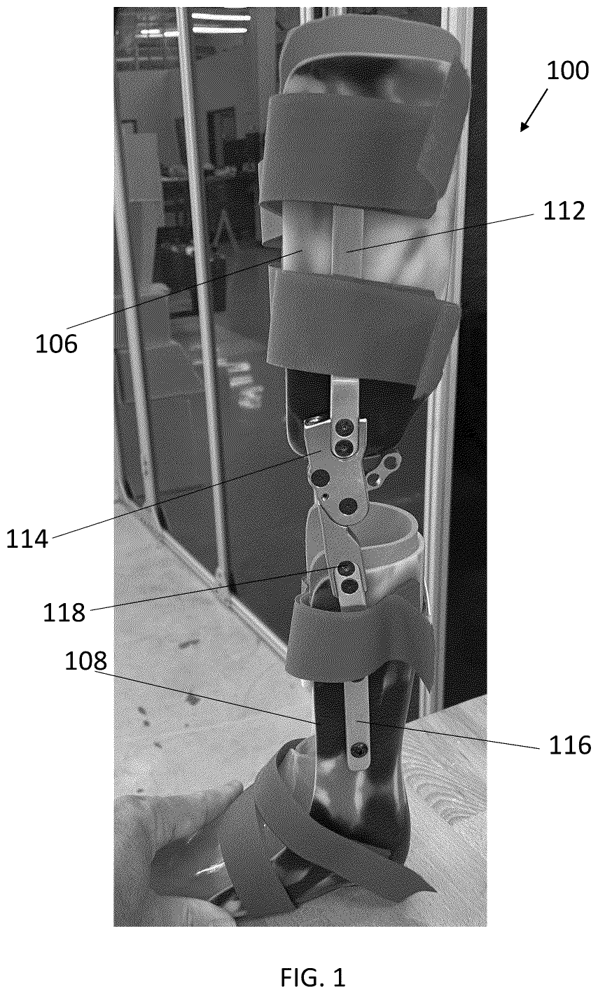

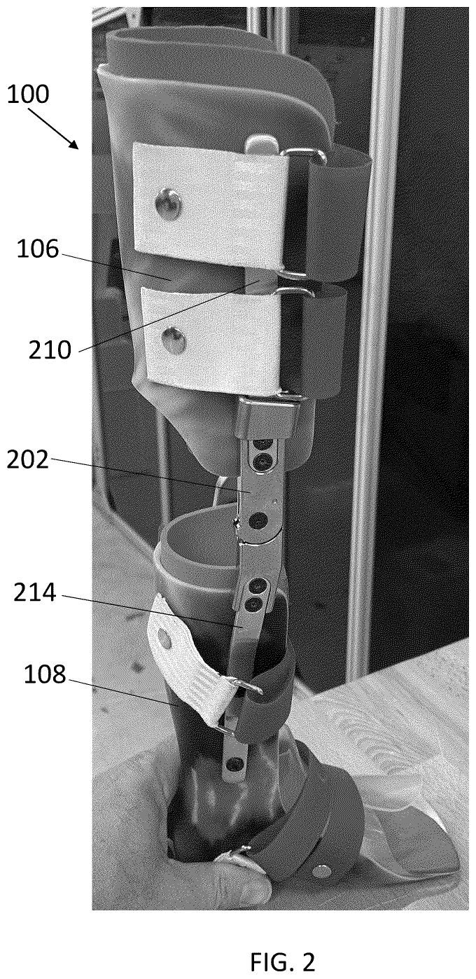

FIG. 1 illustrates a side view of one example of an orthotic knee brace device 100 having a modular joint assembly 104. FIG. 2 illustrates an opposite side view of the brace device 100 with another modular joint assembly 202. While a knee brace is shown and described herein, not all embodiments of the inventive subject matter are limited to knee braces unless explicitly stated to be limited to knee braces. One or more examples of the modular joint assemblies 104, 202 described herein may be used in connection with other types of orthotic braces or devices, such as ankle braces. The modular joint assemblies 104, 202 of the same brace device 100 may be identical or the same, or may differ from each other.

The brace device 100 includes an upper cuff 104 and a lower cuff 106. The upper cuff 104 is body that entirely or partially extends around part of a patient's limb, such as a patient's thigh. With respect to knee braces, the upper cuff 104 can be referred to as a thigh cuff. The lower cuff 106 is another body that entirely or partially extends around part of the patient's limb on a side that is opposite the modular joint assembly 102. For example, the lower cuff 106 can extend around part of a patient's shin, calf, and/or foot. With respect to knee braces, the lower cuff 106 can be referred to as a shin cuff.

The upper cuff 104 can connected with a pair of opposing upper struts 208, 110 on opposite sides of the upper cuff 104. The upper strut 208 can be referred to as an interior or inner upper strut 208 as this strut 208 may extend along the inner part of the patient's thigh (e.g., that faces the patient's other thigh). The upper strut 110 can be referred to as an exterior or outer upper strut 110 as this strut 110 may extend along the outside of the patient's thigh (e.g., that faces away from the patient's body). Optionally, only one of the upper struts 208, 110 is provided.

The lower cuff 106 is connected with a pair of opposing lower struts 212, 114 on opposite sides of the lower cuff 106. The lower strut 212 can be referred to as an interior or inner lower strut 212 as this strut 212 may extend along the inner part of the patient's leg along the fibula (e.g., that faces the patient's other fibula). The lower strut 114 can be referred to as an exterior or outer lower strut 114 as this strut 114 may extend along the outside of the patient's leg along the tibia and fibula (e.g., that faces away from the patient's body). Optionally, only one of the lower struts 212, 114 is provided.

The modular joint assemblies 202, 104 connect pairs of the struts 210, 112, 214, 116 that are on the same side of the patient. For example, the inner modular joint assembly 202 is connected with the inner upper strut 210 and the inner lower strut 214. The outer modular joint assembly 104 is connected with the outer upper strut 112 and the outer lower strut 116. As described herein, different sizes of the modular joint assemblies 202, 104 can be interchanged with the same struts 210, 112, 214, 116 and/or modular joint assemblies 202, 104 having different operating modes can be interchanged with the same struts 210, 112, 214, 116. This can allow for the same struts 210, 112, 214, 116 to be re-used as a patient grows (e.g., by swapping out shorter modular joint assemblies with longer modular joint assemblies), thereby avoiding the time and cost involved with creating new struts 210, 112, 214, 116 for the patient or re-shaping the existing struts 210, 112, 214, 116 for the patient. This also can allow for the same struts 210, 112, 214, 116 to be re-used while the center of the modular joint assembly 104, 202 changes. The center of the modular joint assembly 104, 202 may be the pivot axis (described below) about which the knee or ankle may pivot or rotate. The center of the modular joint assembly 104, 202 may need to be changed due to a prior misalignment of the modular joint assembly 104, 202, growth of the patient, or the like. This can be done by swapping out different arms of the modular joint assemblies 104, 202, as described herein.

Additionally, the modular joint assemblies can be swapped with other modular joint assemblies that operate in different ways. For example, a ring lock modular joint assembly can be swapped out with a ratcheting modular joint assembly, a drop lock modular joint assembly, a spring assisted modular joint assembly, a free motion modular joint assembly, or a ratcheted modular joint assembly, or vice-versa. The modular joint assemblies may be swapped out with each other by merely replacing a few fasteners 118 that connect the modular joint assemblies with the struts 210, 112, 214, 116. These fasteners 118 can be screws, pins, bolts, or the like.

In the illustrated example, the inner modular joint assembly 202 visible in FIG. 2 is a ratcheting modular joint assembly and the outer modular joint assembly 104 visible in FIG. 1 is a drop lock modular joint assembly. The same or different types of modular joint assemblies may be used in the same brace device 100. For example, both modular joint assemblies in a brace device may be ratcheting modular joint assemblies, drop lock modular joint assemblies, free motion modular joint assemblies, spring assisted modular joint assemblies, etc. As another example, the inner modular joint assembly may be one of a ratcheting modular joint assembly, a drop lock modular joint assembly, a free motion modular joint assembly, a spring assisted modular joint assembly, etc., while the outer modular joint assembly is a different modular joint assembly (such as another one of the ratcheting modular joint assembly, the drop lock modular joint assembly, the free motion modular joint assembly, the spring assisted modular joint assembly, etc., that is different from the inner modular joint assembly).

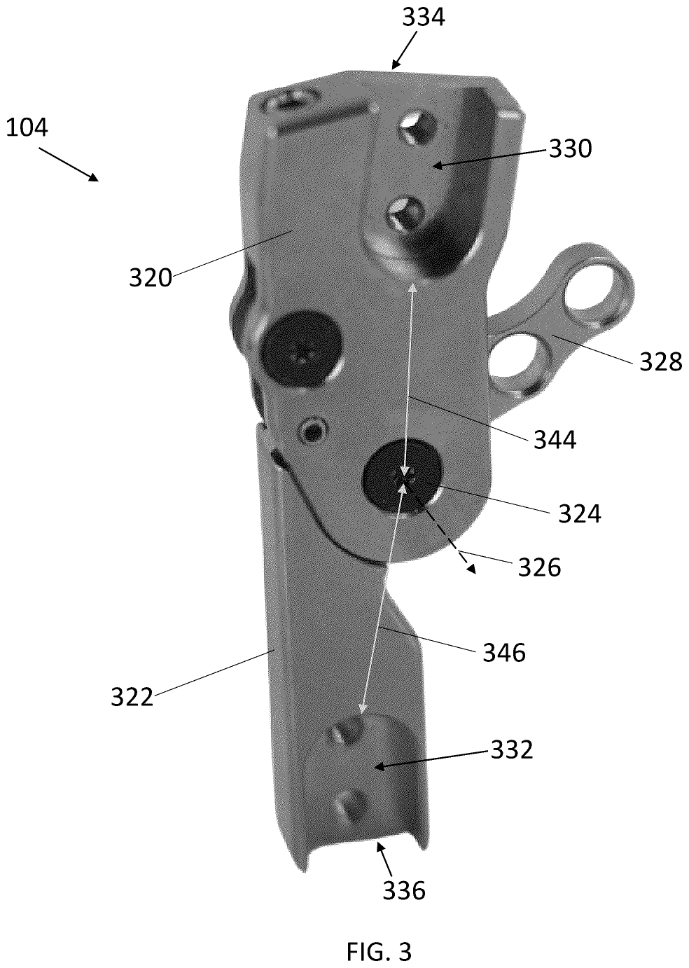

FIG. 3 illustrates one example of the ratcheting modular joint assembly 104 shown in FIG. 1. The ratcheting modular joint assembly 104 includes an upper arm 320 and a lower arm 322 that are connected to each other and that can pivot relative to each other. The upper and lower arms 320, 322 are connected with each other by one or more fasteners 324, such as pins, screws, or the like. The fasteners 324 that connect the arms 320, 322 may be the same or different as the fasteners 118. One of the fasteners 324 may be located at or coaxial with a pivot axis 326 of the modular joint assembly 104. The arms 320, 322 pivot relative to each other about or around this pivot axis 326. For example, as the patient wearing the knee brace 100 (shown in FIG. 1) walks, the arms 320, 322 may pivot about or around the pivot axis 326 to coincide with the movements of the patient's thigh and shin.

The modular joint assembly 104 may include a ratchet inside the arms 320, 322. For example, one of the arms 320 or 322 may include an interior gear or rack (e.g., a toothed wheel with each tooth having a slanted side and an opposite side that extends along a radial direction from a center of the gear or rack, or from the pivot axis 326). The other of the arms 322 or 320 may include an interior pawl (e.g., a spring loaded end of a release lever 328 that is inside the arm 322 or 320) that engages the teeth of the gear or rack. During pivoting of the arms 320, 322 in one direction, the pawl may move over the slanted or angled sides of the teeth of the gear. The arms 320, 322 can rotate or pivot in this direction. During this pivoting, the pawl can slide up and over the slanted side of a tooth of the gear and drop down the opposite radially extending side of the same tooth of the gear. If the arms 320, 322 attempt to then rotate in an opposite direction, the pawl engages the radially extending side of a tooth and prevents rotation or pivoting of the arms 320, 322 in this direction. The patient can press on the release lever 328 to lift or move the pawl away and over the radially extending side of the tooth to disengage the pawl from the gear. This can allow the arms 320, 322 to freely pivot in either direction until the release lever 328 is released.

A biasing element (e.g., a spring) inside the modular joint assembly 104 can bias the release lever 328 in a direction that causes the pawl to move inward toward the gear so that the pawl engages the radially extending side of each tooth as the pawl moves over each tooth. The patient can press or pull on the release lever 328 to overcome the bias of the biasing element on the pawl to allow the arms 320, 322 to freely pivot relative to each other in either direction.

Each of the arms 320, 322 includes a strut recess 330 or 332 in the illustrated example. The upper arm 320 can include the strut recess 330 and the lower arm can include the strut recess 332. These recesses 330, 332 inwardly extend from distal ends 334, 336 of the arms 320, 322. Each arm 320, 322 is elongated and has a length dimension 344, 346 that extends along the length of the arm 320, 322 from the recess 330, 332 to the pivot axis 326 (e.g., the nearest part of the recess 330, 332 to the pivot axis 326).

The recesses 330, 332 are open on one side of the modular joint assembly 104 (e.g., the outer side, or the side that faces away from the patient's leg) in the illustrated example. Alternatively, the recesses 330, 332 may be open on the other side of the modular joint assembly 104 (e.g., the inner side, or the side that faces the patient's leg). In another example, the recesses 330, 332 may be formed as conduits that extend into the distal ends 334, 336 of the arms 320, 322 without being exposed or open on either side of the arms 320, 322. The recesses 330, 332 provide space for the receiving the struts 210, 112, 214, 116. This can reduce the overall size of the brace device 100 and the modular joint assembly 104 (relative to attaching the struts 210, 112, 214, 116 to the arms that do not have the recesses 330, 332 or outside of the recesses 330, 332).

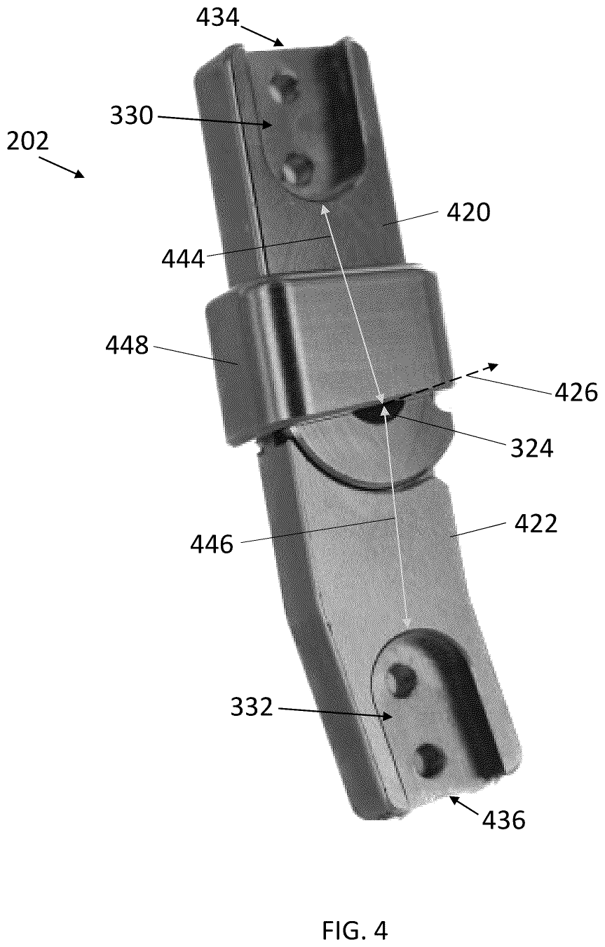

FIG. 4 illustrates one example of the drop lock modular joint assembly 202 shown in FIG. 2. The drop lock modular joint assembly 202 includes an upper arm 420 and a lower arm 422 that are connected to each other and that can pivot relative to each other. The upper and lower arms 420, 422 are connected with each other by one or more of the fasteners 324. The fasteners 324 shown in FIG. 4 may be located at or coaxial with a pivot axis 426 of the modular joint assembly 202. The arms 420, 422 pivot relative to each other about or around this pivot axis 426.

Each of the arms 420, 422 includes one of the strut recesses 330 or 332. Similar to the arms 320, 322 shown in FIG. 3, the recesses 330, 332 inwardly extend from distal ends 434, 436 of the arms 420, 422. Each arm 420, 422 is elongated and has a length dimension 444, 446 that extends along the length of the arm 420, 422 from the recess 330, 332 of that arm 420, 422 to the pivot axis 426 (e.g., from the closest point of the recess 330, 332 to the pivot axis 426). The recesses 330, 332 are open on one side of the modular joint assembly 202, or may be open on the other side of the modular joint assembly 202, or may be formed as conduits that extend into the distal ends 434, 436 of the arms 420, 422 without being exposed or open on either side of the arms 420, 422. As described above, the recesses 330, 332 provide space for the receiving the struts 210, 112, 214, 116.

The modular joint assembly 202 may include an annular body 448, such as a ring, that extends around or encircles the upper arm 420. This annular body 448 may freely slide along (e.g., up and down) the upper arm 420. As shown in FIG. 4, the lower arm 422 may partially extend into the upper arm 420 or parts of the upper arm 420 and the lower arm 422 may extend past the other so that the pivot axis 426 extends through each of the upper and lower arms 420, 422. The annular body 448 may slide down to the position shown in FIG. 4. This may occur when the patient stands up so that gravity can pull the annular body 448 down the length of the upper arm 420.

The annular body 448 may stop in a location in which the annular body 448 encircles part of the upper arm 420 and part of the lower arm 422, as shown in FIG. 4. In this location, the annular body 448 prevents pivoting of the arms 420, 422 relative to each other. This can lock the brace 100 and prevent the brace 100 from pivoting about or around the pivot axis 426. This can be useful in assisting patient's in keeping their joint within the brace 100 straight, such as when the patient needs assistance standing upright. The patient can manually slide the annular body 448 upward so that the annular body 448 is no longer over both arms 420, 422 of the modular joint assembly 202 and thereby allows the arms 420, 422 to pivot relative to each other. This permits the patient to bend their leg again.

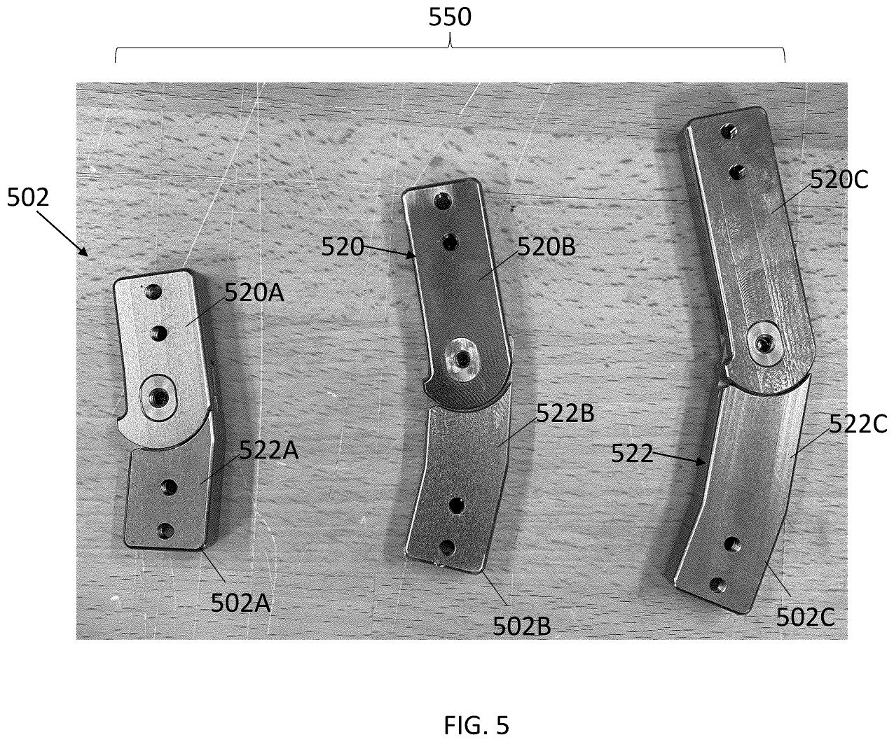

FIG. 5 illustrates one example of a set or kit 550 of modular joint assemblies 502 (e.g., modular joint assemblies 502A-C). The set or kit 550 may be used by a healthcare provider that is providing and/or adjusting the brace device 100 for a patient. The set or kit 550 can include more or fewer modular joint assemblies 502A-C than is shown in FIG. 5. Each of the modular joint assemblies 502 in the set or kit 550 can represent either the modular joint assembly 104 or 202, or another modular joint assembly, but with upper arms 520 (e.g., arms 520A-C) and/or lower arms 522 (e.g., arms 522A-C) that are differently sized from each other.

The upper arms 520A-C of different modular joint assemblies 502 may have different length dimensions 344 (shown in FIG. 3) from each other and/or the lower arms 522A-C of the modular joint assemblies 502 may have different length dimensions 346 (shown in FIG. 3) from each other. This can allow for the modular joint assemblies 502A-C to be swapped out with each other as a patient grows. For example, as a young patient's legs become longer with age and growth, the shorter modular joint assemblies 502A in the brace device 100 may be disconnected from the upper struts 112 or 210 (shown in FIGS. 1 and 2) and from the lower struts 116 or 214 (shown in FIGS. 1 and 2). The fasteners 118 (shown in FIG. 1) connecting the arms 520A, 522A of the modular joint assembly 502A can be removed, the upper and lower struts 112, 210; 116, 214 can be separated from the arms 520A, 522A, another modular joint assembly 502B or 502C can be selected by the healthcare provider, the struts 112, 210; 116, 214 can be placed into the recesses 330, 332 (shown in FIG. 3) in the arms 520B/520C, 522B/522C of the replacement modular joint assembly 502B or 502C, and the fasteners 118 can be inserted into the struts 112, 210; 116, 214 and arms 520B/520C, 522B/522C of the replacement modular joint assembly 502B or 502C to replace the modular joint assembly 502A with the modular joint assembly 502B or 502C.

Additionally, some patients may grow in such a way that the upper arm 520 of one modular joint assembly 502 can be swapped out with a shorter or longer upper arm 520 or another modular joint assembly 502, and the lower arm 520 can either be kept and not swapped out, or swapped out with yet another modular joint assembly 502. For example, a patient's legs may grow such that replacing the modular joint assembly 502A with the modular joint assembly 502C would result in the pivot axis 326, 426 (shown in FIGS. 3 and 4) being misaligned from one or more axes of rotation of the knee or ankle in the brace device 100. This can cause significant irritation to the knee joint or ankle joint, discomfort to the patient, and/or prevent the knee joint or ankle joint from bending properly. Instead, the upper and lower arms 520, 522 from different modular joint assemblies 502 may be combined with each other and used in the brace device 100.

For example, a patient may currently have the brace device 100 with the modular joint assembly 502A having the shortest arms 520A, 522A in the set or kit 550. Once the patient grows too much to continue using the modular joint assembly 502A, the modular joint assembly 502A may be replaced with a hybrid length modular joint assembly 502. This hybrid length modular joint assembly 502 may include the lower arm 522B of the middle length modular joint assembly 502B and the upper arm 520C of the longest modular joint assembly 502C. The fastener 324 (shown in FIG. 3) connecting the arms 520B, 522B of the middle length modular joint assembly 502B and the fastener 324 connecting the arms 520C, 522C of the longer length modular joint assembly 502C may be removed. The lower arm 522B of the middle length modular joint assembly 502B and the upper arm 520C of the longer length modular joint assembly 502C can be connected with each other, and a fastener 324 placed to connect the arms 520C, 522B with each other to form the hybrid modular joint assembly 502. This can give healthcare providers a wide variety of differently sized arms 520, 522 to form different modular joint assemblies 502 to ensure that the pivot axis 326, 426 is aligned with the patient's knee or ankle. Swapping out the differently sized arms among the kit to account for patient growth while maintaining alignment of the joint assemblies with the joint can avoid the costly and time-consuming need to repeatedly re-shape or re-form the struts.

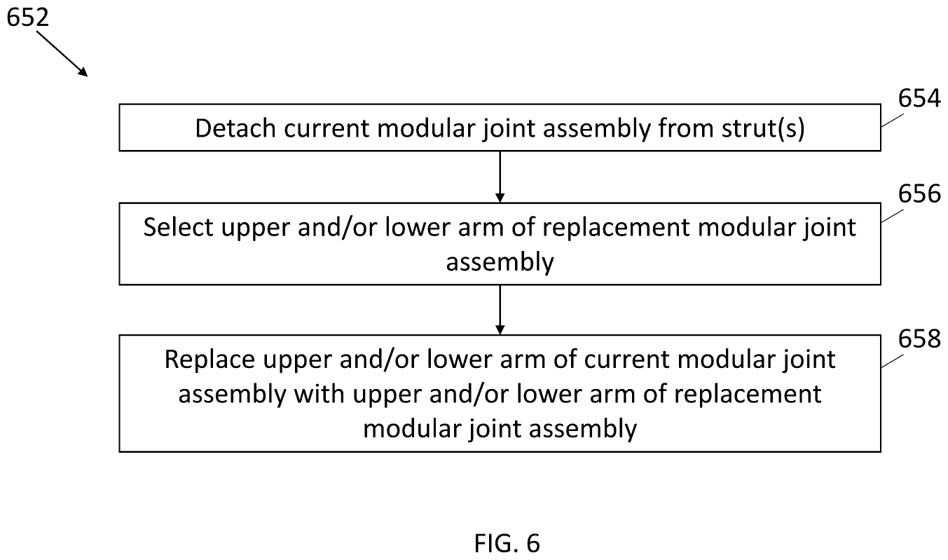

FIG. 6 illustrates a flowchart of one example of a method 652 for fitting a brace device to a patient. The method 652 can be used to fit or replace part or all of a modular joint assembly 104, 202, 502 of the brace device 100 with part or all of another modular joint assembly 104, 202, 502 to ensure that the brace device 100 continues to fit the patient as the patient grows. At 654, a modular joint assembly that is currently connected to the struts of the brace device may be disconnected from the struts. If the entire modular joint assembly is being replaced, then the upper and lower struts connected to the upper and lower arms, respectively, of the modular joint assembly may be separated from each other. Optionally, if only the upper arm of the modular joint assembly is being replaced, then the upper strut connected to the upper arm of the modular joint assembly may be separated from each other. If only the lower arm of the modular joint assembly is being replaced, then the lower strut connected to the lower arm of the modular joint assembly may be separated from each other. Additionally, if only one of the arms is being replaced, then the arm being replaced can be separated from the other arm.

At 656, an upper and/or lower arm of a replacement modular joint assembly is selected. For example, a longer or shorter arm may be selected to replace the shorter or longer arm of the current modular joint assembly. This may be needed to ensure that the pivot axis of the replacement modular joint assembly is aligned with rotation of the knee or ankle while the patient is wearing the brace device. Optionally, both the upper and lower arms may be replaced.

At 658, the upper and/or lower arm of the current modular joint assembly is replaced with the upper and/or lower arm of the replacement modular joint assembly. For example, the replacement arm(s) can be connected to the struts of the brace device. Using such a modular joint assembly can allow for easier modifications of the brace device to ensure that the pivot axis of the modular joint assembly is aligned with the axis of rotation of the knee, such as the flexion-extension (FE) axis.

Use of phrases such as “one or more of . . . and,” “one or more of . . . or,” “at least one of . . . and,” and “at least one of . . . or” are meant to encompass including only a single one of the items used in connection with the phrase, at least one of each one of the items used in connection with the phrase, or multiple ones of any or each of the items used in connection with the phrase. For example, “one or more of A, B, and C,” “one or more of A, B, or C,” “at least one of A, B, and C,” and “at least one of A, B, or C” each can mean (1) at least one A, (2) at least one B, (3) at least one C, (4) at least one A and at least one B, (5) at least one A, at least one B, and at least one C, (6) at least one B and at least one C, or (7) at least one A and at least one C.

As used herein, an element or step recited in the singular and preceded with the word “a” or “an” do not exclude the plural of said elements or operations, unless such exclusion is explicitly stated. Furthermore, references to “one embodiment” of the invention do not exclude the existence of additional embodiments that incorporate the recited features. Moreover, unless explicitly stated to the contrary, embodiments “comprising,” “comprises,” “including,” “includes,” “having,” or “has” an element or a plurality of elements having a particular property may include additional such elements not having that property. In the appended claims, the terms “including” and “in which” are used as the plain-English equivalents of the respective terms “comprising” and “wherein.” Moreover, in the following claims, the terms “first,” “second,” and “third,” etc. are used merely as labels, and do not impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function devoid of further structure.

This written description uses examples to disclose several embodiments of the subject matter, including the best mode, and to enable one of ordinary skill in the art to practice the embodiments of subject matter, including making and using any devices or systems and performing any incorporated methods. The patentable scope of the subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A modular joint assembly for a brace device for a joint, the modular joint assembly comprising:
an upper arm configured to be fastened to an upper leg strut of the brace device; and
a lower arm configured to be fastened to a lower leg strut of the brace device, the upper arm and the lower arm connected with each other and configured to pivot relative to each other about a pivot axis of the brace device,
wherein one or both of the upper arm or the lower arm is configured to be separated from another of the upper arm or the lower arm and replaced with a longer or shorter arm to change a location of the pivot axis in the brace device.

2. The modular joint assembly of claim 1, wherein the one or both of the upper arm or the lower arm is configured to be separated from another of the upper arm or the lower arm and replaced with the longer or shorter arm while being connected to the same upper leg strut or the same lower leg strut in the brace device.

3. The modular joint assembly of claim 1, wherein the upper arm is configured to be separated from the lower arm and replaced with a longer upper arm to maintain the pivot axis in alignment with a knee of a patient wearing the brace device.

4. The modular joint assembly of claim 1, wherein the lower arm is configured to be separated from the upper arm and replaced with a longer lower arm to maintain the pivot axis in alignment with a knee of a patient wearing the brace device.

5. The modular joint assembly of claim 1, wherein the upper arm and the lower arm include an internal ratchet to restrict permit pivoting of the upper arm or the lower arm in a first direction about the pivot axis and prevent pivoting of the upper arm or the lower arm in an opposite second direction about the pivot axis.

6. The modular joint assembly of claim 1, wherein the upper arm includes an annular body configured to lower and lock the upper arm to the lower arm and prevent pivoting of the upper arm or the lower arm relative to each other.

7. A kit for adjusting a brace device for a joint, the kit comprising:
plural modular joint assemblies each having an upper arm configured to be fastened to an upper leg strut of the brace device and a lower arm configured to be fastened to a lower leg strut of the brace device, the upper arm and the lower arm connected with each other at a pivot axis about which the upper arm and the lower arm pivot relative to each other,
each of the modular joint assemblies in the kit having a differently sized upper arm, a differently sized lower arm, or both the differently sized upper arm and the differently sized lower arm than the other modular joint assemblies in the kit,
wherein one or both of the upper arm or the lower arm of each of the modular joint assemblies is configured to be separated from another of the upper arm or the lower arm of the same modular joint assembly and replaced with a longer or shorter arm of another one of the modular joint assemblies to change a location of the pivot axis in the brace device.

8. The kit of claim 7, wherein the one or both of the upper arm or the lower arm of each modular joint assembly is configured to be separated from another of the upper arm or the lower arm of the same modular joint assembly and replaced with the longer or shorter arm of the other one of the modular joint assemblies while being connected to the same upper leg strut or the same lower leg strut in the brace device.

9. The kit of claim 7, wherein the upper arm of a first modular joint assembly of the modular joint assemblies is configured to be separated from the lower arm of the first modular joint assembly and replaced with a longer upper arm of a second modular joint assembly of the modular joint assemblies to maintain the pivot axis in alignment with a knee of a patient wearing the brace device.

10. The kit of claim 7, wherein the lower arm of a first modular joint assembly of the modular joint assemblies is configured to be separated from the upper arm of the first modular joint assembly and replaced with a longer lower arm of a second modular joint assembly to maintain the pivot axis in alignment with a knee of a patient wearing the brace device.

11. The kit of claim 7, wherein the upper arm and the lower arm of at least one of the modular joint assemblies include an internal ratchet to restrict permit pivoting of the upper arm or the lower arm in a first direction about the pivot axis and prevent pivoting of the upper arm or the lower arm in an opposite second direction about the pivot axis.

12. The kit of claim 7, wherein the upper arm of at least one of the modular joint assemblies includes an annular body configured to lower and lock the upper arm to the lower arm and prevent pivoting of the upper arm or the lower arm relative to each other.

13. A method comprising:
disconnecting a first upper arm or a first lower arm of a first modular joint assembly in a brace device for a joint from an upper strut or a lower strut of the brace device, the upper strut connected to an upper leg cuff, the lower strut connected to a lower leg cuff;
obtaining a longer or shorter second upper arm or a second lower arm; and
connecting the longer or shorter second upper arm or the second lower arm to the upper strut or the lower strut to change a location of a pivot axis of the first modular joint assembly or maintain alignment of the pivot axis of the first modular joint assembly with a rotation axis of a joint in the brace device.

14. The method of claim 13, wherein both the first upper arm is disconnected from the upper strut and the first lower arm is disconnected from the lower strut, both the second upper arm and the second lower arm are obtained, and both the second upper arm is connected with the upper strut and the second lower arm is connected with the lower strut to replace the first modular joint assembly with a second modular joint assembly.

15. The method of claim 13, wherein the first modular joint assembly is a drop lock modular joint assembly, and the second modular joint assembly is a ratcheting modular joint assembly.

16. The method of claim 13, wherein the first modular joint assembly is a ratcheting modular joint assembly, and the second modular joint assembly is a drop lock modular joint assembly.

17. The method of claim 13, wherein the first upper arm is disconnected from the upper strut while the first lower arm remains connected to the lower strut, only the second upper arm is obtained, and only the second upper arm is connected with the upper strut with the first lower arm remaining connected to the lower strut to replace the first upper arm of the first modular joint assembly.

18. The method of claim 13, wherein the first lower arm is disconnected from the lower strut while the first upper arm remains connected to the upper strut, only the second lower arm is obtained, and only the second lower arm is connected with the lower strut with the first upper arm remaining connected to the upper strut to replace the first lower arm of the first modular joint assembly.

19. The method of claim 13, wherein the longer or shorter second upper arm or the second lower arm is connected to the upper strut or the lower strut to change the location of the pivot axis of the first modular joint assembly or maintain alignment of the pivot axis of the first modular joint assembly with the rotation axis of the joint in the brace device without having to replace the upper strut or the lower strut.

20. The method of claim 13, wherein the longer or shorter second upper arm or the second lower arm is connected to the upper strut or the lower strut to change the location of the pivot axis of the first modular joint assembly or maintain alignment of the pivot axis of the first modular joint assembly with the rotation axis of the joint in the brace device without having to re-shape the upper strut or the lower strut.
